(19) United States
(12) Patent Application Publication
(10) Pub. No.: US 2023/0125117 A1
(43) Pub. Date: Apr. 27, 2023

(54) HOT AIR CIRCULATING FOOD WASTE DRYER

(71) Applicant: Loofen Co., Ltd., Uiwang-si, Gyeonggi-do (KR)

(72) Inventor: Hee Ja Lee, Yongin-si, Gyeonggi-do (KR)

(73) Assignee: Loofen Co., Ltd., Uiwang-si, Gyeonggi-do (KR)

(21) Appl. No.: 17/618,867

(22) PCT Filed: Jan. 31, 2020
(86) PCT No.: PCT/KR2020/001547

§ 371 Date: Feb. 18, 2022

(30) Foreign Application Priority Data
Jul. 17, 2019 (KR) 10-2019-0086435

(51) Int. Cl. B09B 3/40; F26B 9/06; F26B 21/00; F26B 21/10; F26B 25/00; B01D 53/04; B01J 20/20; B01J 20/18; B01J 20/02; B01J 20/04; A61L 9/014; A61L 11/00

(52) U.S. Cl. CPC B09B 3/40; F26B 9/06; F26B 21/004; F26B 21/10; F26B 25/005; B01D 53/04; B01J 20/20; B01J 20/18; B01J 20/0237; B01J 20/0222; B01J 20/041; A61L 9/014; A61L 11/00; F26B 2200/04; B09B 2101/70

(57) ABSTRACT

The present invention relates to a food waste dryer comprising: a main body in which a drying space is formed so that a portion thereof is open to the outside; a front door for closing the open portion of the main body; a drying basket which is disposed in the drying space of the main body and in which food is accommodated; an air circulation unit which includes a heater capable of heating the air of the drying space, and which suctions the air inside the drying space, and then blows, at the food waste, the air heated by the heater so as dry the food waste; a circulation pipe capable of discharging, to the outside through an exhaust pipe, the air that includes steam vaporized during a food waste drying process, and recirculating, to the drying space through a blowing pipe, the air heated by the heater; and a deodorizing device mounted on the outside of the main body so as to adsorb odor particles from the air discharged to the outside through the circulation pipe, thereby enabling the air to be deodorized, wherein the deodorizing device includes a deodorant comprising activated carbon and zeolite. According to the present invention, a fixed amount of steam, which is generated by vaporization and evaporation during the food waste drying process, is maintained, and the adsorption and deodorization performance of the odor particles can be improved.

### TECHNICAL FIELD

The present invention relates to a food waste dryer, and more particularly, to a food waste dryer capable of maintaining a constant amount of water vapor that is generated through vaporization and evaporation during the drying process for food waste and improving adsorption and deodorization performances for odor particles.

### BACKGROUND ART

In general, food waste generated at home is mainly treated through a landfill method, causing serious environmental pollution, and furthermore, since the food waste decays with a severe odor, the food waste is regarded as a main factor that pollutes the surrounding environment as well as the overall living environment.

In order to solve the above problem, food waste handlers and food waste dryers have been developed. Conventional food waste handlers treat food wastes through a fermentation method using microorganisms, so the conventional food waste handlers have advantages that the convenience of food waste disposal is excellent, but the conventional food waste handlers represent some difficulties in maintaining and managing the microorganisms, and there is a problem that the processing speed for the food waste is slow because they are entirely depending on the microorganisms.

The conventional food waste dryer is a device for drying the food waste by wind or heat, and the dried food waste is used as fertilizer or buried through a landfill method.

Japanese Unexamined Patent Application No. 1997-159358 has been suggested as an example of a conventional technique related to the food waste dryer. According to the above technique, a blower is disposed above a storage box accommodated inside a dryer, and an air circulation path is formed inside the dryer to circulate air from the blower the food waste.

Accordingly, a circulating air flow generated by the blower and the air circulation path repeatedly widely dries a surface layer of the food waste, thereby increasing the drying efficiency compared to the natural drying.

However, the food waste dryer according to the related art does not have a separate heating wire or heater, so there is a problem in that the drying processing speed for the food waste is relatively low. In addition, since it is necessary to ventilate the odor of food waste through a drain due to the lack of a separate deodorizing device, there is a limitation in installation places because it can be installed only around the sink. Further, since the odor of discharged waste food often flows back along the drain, the number of complaints caused by the odor is increased.

### DISCLOSURE

### Technical Problem

The present invention has been suggested to solve the above problem, and it is an object of the present invention to provide a food waste dryer having an improved structure, capable of maintaining a constant amount of water vapor that is generated through vaporization and evaporation during the drying process for food waste and improving adsorption and deodorization performances for odor particles.

### Technical Solution

In order to accomplish the above object, a food waste dryer according to the present invention includes: a body in which a drying space is formed so that a portion thereof is open to an outside; a front door for closing the open portion of the body; a drying basket which is disposed in the drying space of the body and in which food waste is accommodated; an air circulation unit including a heater adapted for heating air inside the drying space, in which the air circulation unit suctions the air inside the drying space and then blows the air heated by the heater to the food waste so as to dry the food waste; a circulation pipe configured to discharge air that includes water vapor vaporized during a food waste drying process to an outside through an exhaust pipe, and to recirculate air heated by the heater to the drying space through a blower pipe; and a deodorizing device mounted on an outside of the body so as to deodorize air by adsorbing odor particles from the air discharged to the outside through the circulation pipe, wherein the deodorizing device includes a deodorant including activated carbon and zeolite.

Preferably, a ratio of a flow rate of the air recirculated to the drying space through the blower pipe to a flow rate of the air discharged to the outside through the exhaust pipe is 1:3 to 6.

Preferably, the deodorant includes 5 to 15 parts by weight of zeolite based on 100 parts by weight of activated carbon.

Preferably, the deodorant includes activated carbon containing copper (Cu), manganese (Mn), sodium hydroxide (NaOH), and potassium hydroxide (KOH).

Preferably, the deodorant is obtained by dissolving 5,000 to 80,000 mg of manganese (Mn), 5,000 to 200,000 mg of copper (Cu), 100 to 10,000 mg of sodium hydroxide (NaOH), and 100 to 10,000 mg of potassium hydroxide (KOH) in 1 [symbol] of water, adding activated carbon having an average particle size of 0.5 to 1.5 mm and zeolite having an average particle size of 2 to 7 mm at a normal temperature, and dehydrating and drying the resultant at a temperature of 90 to 120° C.

Preferably, a UV lamp installed inside the body to sterilize the food waste is further provided.

Preferably, a temperature sensor adapted for detecting a temperature of the drying space is further provided and an operation of the heater is stopped when the temperature of the drying space detected by the temperature sensor exceeds a predetermined temperature.

### Advantageous Effects

According to the present invention, the food waste dryer includes: a body in which a drying space is formed so that a portion thereof is open to an outside; a front door for closing the open portion of the body; a drying basket which is disposed in the drying space of the body and in which food waste is accommodated; an air circulation unit including a heater adapted for heating air inside the drying space, in which the air circulation unit suctions the air inside the drying space and then blows the air heated by the heater to the food waste so as to dry the food waste; a circulation pipe configured to discharge air that includes water vapor vaporized during a food waste drying process to an outside through an exhaust pipe, and to recirculate air heated by the heater to the drying space through a blower pipe; and a deodorizing device mounted on an outside of the body so as to deodorize air by adsorbing odor particles from the air discharged to the outside through the circulation pipe, wherein the deodorizing device includes a deodorant including activated carbon and zeolite. Thus, it is possible to maintain a constant amount of water vapor that is generated through vaporization and evaporation during the drying process for the food waste and the adsorption and deodorization performances for odor particles can be improved by the deodorant.

### DESCRIPTION OF DRAWINGS

FIG. 1 is a perspective view of a food waste dryer according to an embodiment of the present invention.

FIG. 2 is a view showing a state in which a front door of a food waste dryer shown in FIG. 1 is open.

FIG. 3 is a partially cut-away view of a food waste dryer shown in FIG. 1.

FIG. 4 is a vertical-sectional view of a food waste dryer shown in FIG. 1.

FIG. 5 is a view showing a deodorizing device of a food waste dryer shown in FIG. 1.

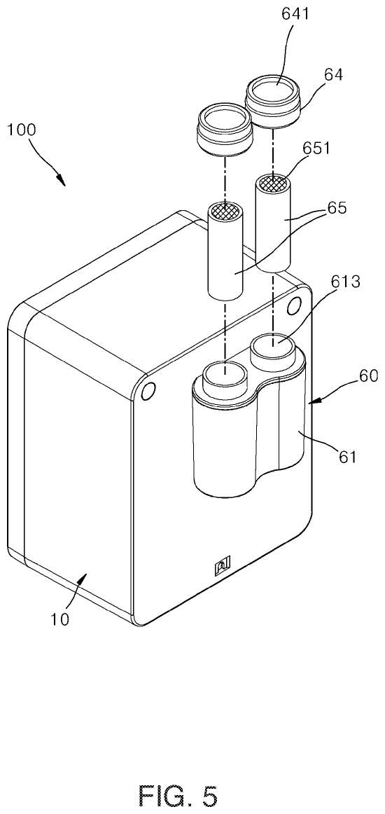

FIG. 6 is a vertical-sectional view of a filter shown in FIG. 5.

### BEST MODE

### Mode for Invention

Hereinafter, preferred embodiments of the present invention will be described in detail with reference to the accompanying drawings.

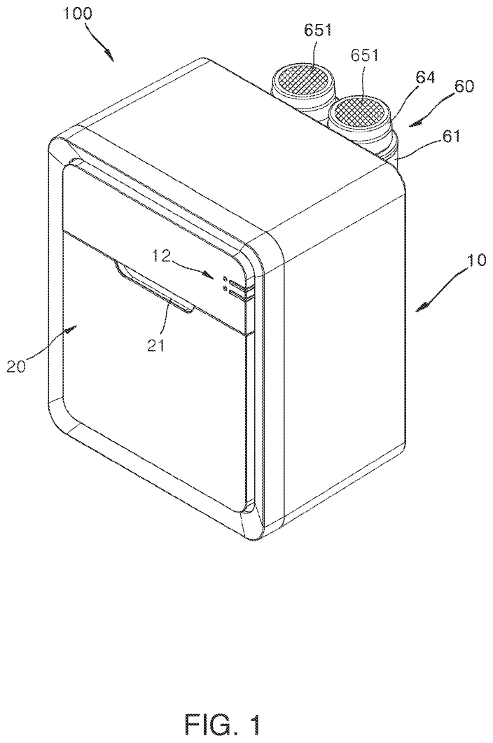

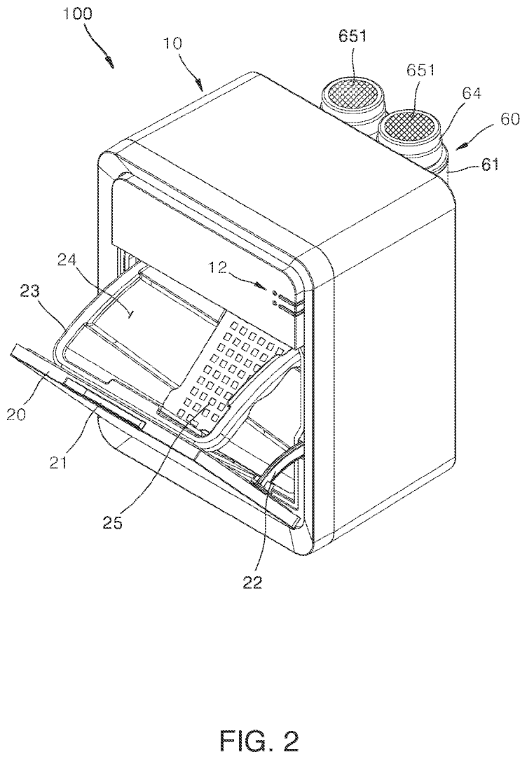

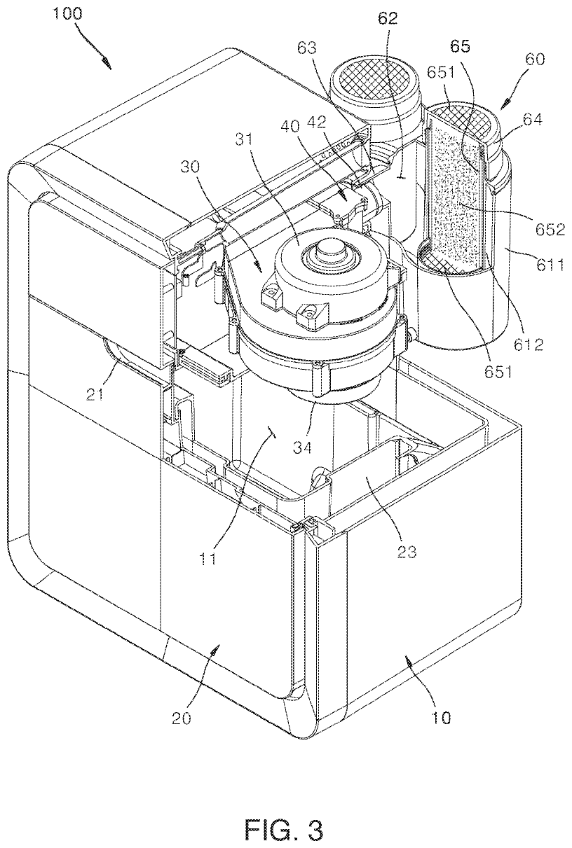

FIG. 1 is a perspective view of a food waste dryer according to an embodiment of the present invention, and FIG. 2 is a view showing a state in which a front door of a food waste dryer shown in FIG. 1 is open. FIG. 3 is a partially cut-away view of a food waste dryer shown in FIG. 1.

Referring to FIGS. 1 to 3, a food waste dryer 100 according to a preferred embodiment of the present invention may be a device capable of drying food waste F using hot air, and include a body 10, a front door 20, an air circulation unit 30, a circulation pipe 40, a UV lamp 50, and a deodorizing device 60.

The body 10 is a case that forms the overall outer appearance of the food waste dryer 100, in which a drying space 11 for drying the food waste F is formed in the body 10 and a part of the drying space 11 is open to the outside.

In the present embodiment, the body 10 is illustrated as a square box shape, in which the drying space 11 is formed in the body 10 and a front surface of the body 10 is open to the outside, but this is an example only. Since an upper surface or a side surface of the body 10 may be open to the outside in use, the shape of the body is not necessarily limited to the above.

A switch unit 12 for operating the food waste dryer 100 may be attached to a front upper end portion of the body 10.

The switch unit 12 may include a power switch, a lamp indicating the operation state of the food waste dryer 100, a switch for driving the UV lamp 50, a lamp for indicating the operation state of the UV lamp 50, and the like.

The body 10 is equipped with a temperature sensor (not shown) capable of detecting the temperature of the drying space 11, and the operation of a heater 33, which will be described below, is stopped when the temperature of the drying space 11 detected by the temperature sensor exceeds a predetermined temperature.

According to the present embodiment, the operation of the heater 33 is stopped when the temperature of the drying space 11 detected by the temperature sensor exceeds 50° C.

The front door 20 is a door that closes an open front portion of the body 10, and includes a grip portion 21, a stopper 22, and a drying basket 23 as shown in FIG. 2.

The front door 20 may cover a part of the open portion of the body 10 to prevent air containing the odor particles generated from the food waste from leaking into the atmosphere in normal times, and the front door 20 may open the inner drying space 11 if necessary so that the food waste can be accommodated in a drying basket 23 to be described below.

The grip portion 21 may be a portion provided at an upper end of the front door 20 to allow a user to grip the grip portion when the user opens or closes the front door 20.

In the present embodiment, the grip portion 21 may be provided in the form of a groove extending left and right.

Since a lower end of the front door 20 is hinged to the body 10, as shown in FIG. 2, the upper end of the front door 20 may be rotated about the lower end when the front door 20 is open.

The stopper 22 may be a restraining device for preventing the front door 20 from being open excessively.

A pair of stoppers 22 may be provided and mounted on both sides of the lower end of the front door 20, respectively.

The drying basket 23 may be a container having a storage space 24 capable of accommodating the food waste F therein, and may be disposed in the drying space 11 of the body 10.

According to the present embodiment, a basket mounting portion 231, to which the drying basket 23 can be hung and mounted, may be formed on the inner surface of the front door 20.

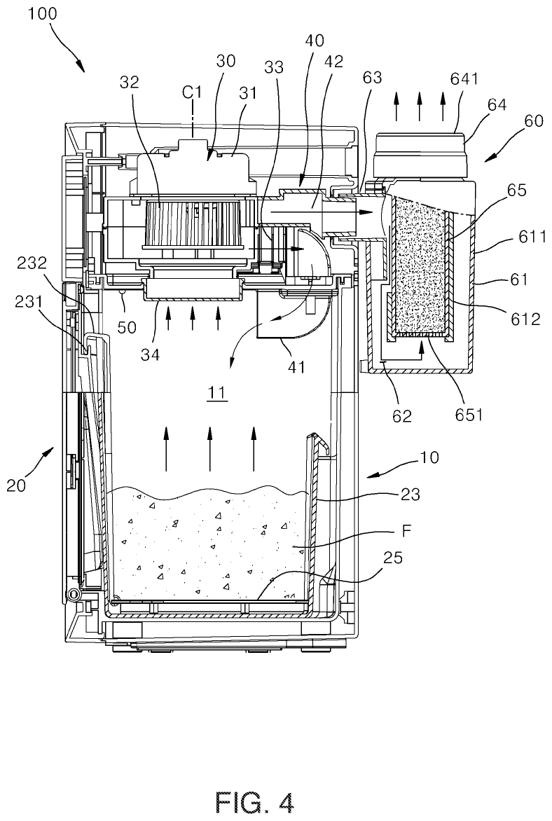

The basket mounting portion 231 may be provided in the form of a groove with an open upper side as shown in FIG. 4.

A hanger portion 232 configured to be detachably hooked to the basket mounting portion 231 may be formed at the upper end of the drying basket 23.

That is, according to the present embodiment, the drying basket 23 has a structure detachably coupled to the inner surface of the front door 20, so that the drying basket 23 may be naturally exposed to the outside when the front door 20 is open as shown in FIG. 2.

A sealing member (not shown) may be attached between the front door 20 and the body 10 to completely isolate the drying space 11 inside the body 10 from the outside when the front door 20 is closed, and to prevent unnecessary noise from being generated when the front door 20 makes contact with the body 10.

As shown in FIGS. 2 and 4, a mesh-type support member 25 may be mounted on the bottom of the drying basket 23.

The mesh-type support member 25 may be a mesh-type member having a plurality of ventilation holes and disposed while being spaced apart from the bottom of the drying basket 23 by a predetermined interval.

Accordingly, a large amount of moisture contained in the food waste F placed on the mesh-type support member 25 may naturally fall to the bottom of the drying basket 23, and only solid ingredients of the food waste F may remain on the mesh-type support member 25, so that it is possible to increase the drying efficiency for the food waste.

In addition, there is a positive effect that the hot air may come into contact with the lower surface of the food waste F placed on the mesh-type support member 25.

Meanwhile, a dielectric sensor (not shown) capable of measuring a change in dielectric constant of the food waste may be mounted inside the drying basket 23.

The dielectric sensor (not shown) may be a sensor for controlling the driving of the heater 33 and an impeller 32, which will be described below.

The dielectric sensor (not shown) may be a sensor for measuring a change in dielectric constant using electromagnetic waves, and may calculate the degree of change in the dielectric relaxation frequency through moisture contained in the food waste to find out the amount of generated water vapor.

That is, if the amount of generated water vapor is small, the dielectric relaxation frequency may increase, so it is determined that there is a large amount of moisture in the food waste and the heater 33 continues to be driven. If the amount of generated water vapor is great, the dielectric relaxation frequency may decrease, so the operation of the heater 33 is stopped.

A high dielectric constant basically means that electric energy is transmitted well. For example, in the soil with a low dielectric constant, electricity does not flow well and electromagnetic waves pass well, but in the case of the soil having a higher dielectric constant due to wetness, electricity flows gradually well, and electromagnetic waves are not easily transmitted.

By mounting and using a dielectric sensor (not shown) using the above property, it is possible to increase the accuracy at a low cost.

Accordingly, by measuring the change in the dielectric constant of the food waste through the dielectric sensor (not shown) to find the amount of generated water vapor, the operation of the heater 33 and the impeller 32 may be controlled. Therefore, the temperature and the amount of wind in the drying space 11 may be adjusted, thereby improving the drying efficiency of the food waste, and simultaneously, the amount of generated water vapor may be kept constant, thereby improving the deodorization performance of the deodorizing device to be described below.

The air circulation unit 30 may be a device that sucks air in the drying space 11, heats the air, and blows the air to the food waste F, and may include a motor 31, the impeller 32, the heater 33, and a suction port 34.

The motor 31 may be an electric motor that rotates about a first central axis Cl by an external power source.

The impeller 32 may be a blower fan that is coupled to the motor 31 and rotates about the first central axis Cl, and may be located below the motor 31 as shown in FIG. 4.

When the impeller 32 rotates, the air in the drying space 11 may be sucked through the suction port 34 provided below the air circulation unit 30, and then the air is blown to the circulation pipe 40.

The heater 33 may be a device for heating the air in the drying space 11 by generating heat by an external power source, and according to the present embodiment, the heater may include a PTC heater (Positive Temperature Coefficient Heater) having a set temperature preferably in the range of about 6070° C.

The PTC heater is widely used in automobiles and the like as a sensor-type heater in which the amount of heat is relatively increased or decreased according to the ambient temperature.

In the present embodiment, the heater 33 may be disposed between the impeller 32 and a blower pipe 41 to be described below.

The suction port 34 may be a hole for sucking air into the air circulation unit 30, and may be communicated with the impeller 32.

In the present embodiment, as shown in FIG. 4, the suction port 34 may be disposed at the upper end of the drying space 11, and may be configured in the form of a mesh net to prevent the food waste, which is boiled by hot air during the drying process for the food waste, from penetrating into the impeller 32.

The circulation pipe 40 may be a pipe for discharging some of the air blown from the impeller 32 to the outside, and recirculating the remaining air to the drying space 11 again. The circulation pipe 40 may include a blower pipe 41 and an exhaust pipe 42.

The blower pipe 41 may be a pipe for recirculating the air, which is blown from the impeller 32 and heated by the heater 33, back to the drying space 11.

One end of the blower pipe 41 may communicate with the impeller 32, and the other end of the blower pipe 41 may communicate with the upper end of the drying space 11.

The exhaust pipe 42 may be a pipe for discharging some of the air blown from the impeller 32 to the outside.

One end of the exhaust pipe 42 may communicates with the impeller 32, and the other end of the exhaust pipe 42 may communicate with the deodorizing device 60.

In the present embodiment, one end of the blower pipe 41 and one end of the exhaust pipe 42 may be disposed adjacent to each other.

Accordingly, the air containing water vapor vaporized in the drying process for the food waste F may be discharged to the outside through the circulation pipe 40, and the air heated by the heater 33 may be recirculated back to the drying space 11.

Here, the ratio of the air flow rate recirculated to the drying space 11 through the blower pipe 41 to the air flow rate discharged to the outside through the exhaust pipe 42 may be preferably set to 1:3 to 6. In the present embodiment, the ratio of the air flow rate is approximately 1:4.

When the ratio of the air flow rate is less than 1:3, there is a problem in that the amount of heat transferred to the food waste F decreases and the overload is applied to the processing capacity of the deodorizing device 60, and when the ratio of the air flow rate exceeds 1:6, the amount of heat transferred to the food waste F increases, but the amount of moisture discharged to the outside decreases, resulting in an increase of energy consumption.

The UV lamp 50 may be an ultraviolet lamp for sterilizing the food waste, and a UV-LED lamp is used in the present embodiment.

As shown in FIG. 4, the UV lamp 50 may be mounted on the upper end of the drying space 11.

In the present embodiment, the UV lamp 50 may be set to be automatically turned off after operating for a predetermined time when the power switch of the food waste dryer 100 is pressed. For example, it can be set to be turned off after initially operating for 5 hours.

The UV lamp 50 may be forcibly operated or stopped by using a separate switch provided in the switch unit 12.

The deodorizing device 60 may be a device capable of adsorbing and deodorizing the odor particles from the air discharged to the outside through the circulation pipe 40, and may be detachably attached to the rear surface of the body 10. The deodorizing device 60 may include a case 61 and a filter 65.

The case 61 may be a case that can accommodate the filter 65, and may include an outer case 611 and an inner case 612.

As shown in FIG. 4, the outer case 611 may be a case arranged to surround the inner case 612.

An inlet 63 communicating with the exhaust pipe 42 may be formed at one end of the outer case 611.

As shown in FIG. 4, the inlet 63 may be detachably coupled to the exhaust pipe 42.

The inner case 612 may be a case disposed inside the outer case 611 while being spaced apart from the outer case 611 by a predetermined distance. In the present embodiment, a pair of inner cases 612 may be provided.

A filter storage hole 613 capable of detachably accommodating the filter 65 may be provided inside the inner case 612.

A lower end of the filter storage hole 613 may support the filter 65, and may be open to allow the air to pass therethrough.

An air passage 62 through which the air discharged from the exhaust pipe 42 can move may be formed in the space between the outer case 611 and the inner case 612.

The air passage 62 may have one end communicating with the inlet 63, and the other end communicating with the lower end of the filter 65.

In a state in which the filter 65 is inserted into the filter storage hole 613, the filter 65 may be fixed by using a plug member 64 such that the filter 65 can be prevented from being separated from the inner case 612.

The plug member 64 may be a member having a hollow 641 therein, and may be screw-coupled to the upper end of the inner case 612.

A mesh member 651 located at the upper end of the filter 65 may be exposed to the outside through the hollow 641.

The filter 65 may be a filter including a deodorant 652 containing activated carbon and zeolite therein.

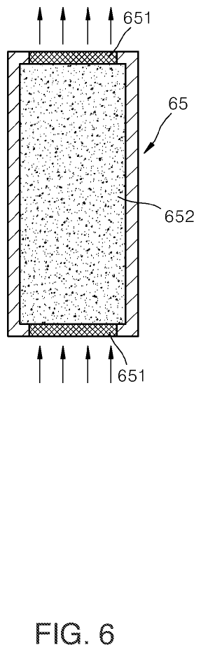

In the present embodiment, as shown in FIG. 6, the filter 65 may be configured in a cylindrical shape and the mesh members 651 through which air can pass may be disposed at upper and lower ends of the filter 65, respectively.

Therefore, the deodorant 652 filled in the filter 65 does not pass through the mesh member 651 and may be maintained in the filter 65.

As shown in FIG. 6, the air in the air passage 62 may be introduced through the lower end of the filter 65, and then discharged to the outside through the upper end of the filter 65 after being deodorized through the deodorant 652.

Since the odor generated from the food waste dryer 100 is derived from the food waste, the odor may have a high moisture content and both acidic and basic properties.

Therefore, the deodorant 652 contained in the deodorizing device 60 needs to have the high instantaneous adsorption performance and high selective adsorption performance in the presence of moisture, and need to adsorb, oxidize and neutralize both basic and acidic odors.

In the present invention, the activated carbon is selected as the deodorant 652, and the average particle size of the activated carbon is set to 0.5 to 1.5 mm in consideration of the smooth flow of exhaust gas and the adsorption capacity of the deodorant 652.

This is because the flow of the exhaust gas is affected when the particle size is less than 0.5 mm so that the internal temperature of the food waste dryer 100 tends to rise, and the flow of the exhaust gas is fast when the particle size exceeds 1.5 mm so that the odor may not be adsorbed, oxidized and/or neutralized well.

In addition, according to the present invention, copper (Cu), manganese (Mn), sodium hydroxide (NaOH) and potassium hydroxide (KOH) may be contained in the activated carbon in use to improve the deodorizing efficiency and to remove the acid odor.

That is, as a result of applying the activated carbon having the above particle size to the food waste dryer 100 according to the present invention, no odor was generated in the initial adsorption, but after a certain time (about 3 days or more) has elapsed, an acidic odor was generated.

For this reason, according to the present invention, metals such as copper (Cu) and manganese (Mn) and neutralizing agents such as sodium hydroxide (NaOH) and potassium hydroxide (KOH) are contained in the activated carbon in use to improve the deodorizing performance and to remove the acid odor.

According to the present invention, manganese (Mn) having a strong oxidizing power may be added in the form of an oxide, and the amount of use thereof is preferably 5,000 to 80,000 mg/ [symbol] . If the amount of manganese is less than 5,000 mg/ [symbol] , the oxidizing power is reduced, and if the amount of manganese exceeds 80,000 mg/ [symbol] , manganese is supersaturated so that it is not dissolved any more.

Copper (Cu) may also be added in the form of an oxide, and have excellent oxidizing power and excellent ammonia deodorizing ability. The amount of use thereof is preferably 5,000 to 200,000 mg/ [symbol] . If the amount of copper is less than 5,000 mg/ [symbol] , the deodorizing ability is lowered, and if the amount of copper exceeds 200,000 mg/ [symbol] , there is no effect of addition.

Sodium hydroxide (NaOH) and potassium hydroxide (KOH) may be added for the purpose of neutralizing an acidic odor, and the amount of use thereof is preferably 100 to 10,000 mg/ [symbol] , respectively. If the amount of sodium hydroxide and potassium hydroxide is less than 100 mg/ [symbol] , the neutralizing function is weak, and if the amount of sodium hydroxide and potassium hydroxide exceeds 10,000 mg/ [symbol] , there is no effect of addition.

As described above, the deodorant 652 according to the present invention may include the activated carbon that contains manganese and copper, which are metals having a very strong oxidizing power and excellent deodorizing ingredients, and sodium hydroxide and potassium hydroxide serving as a neutralizing agent, so that the deodorizing performance can be improved and the acid odor can be effectively removed.

The amount of use of the activated carbon is proportional to the amount of the food waste to be treated. For example, when 100 g of the general food waste is dried, the amount of use of the activated carbon is preferably about 100 to 500 g, but the amount of use may vary depending on the type of the food waste.

According to the present embodiment, the deodorant 652 may be obtained by dissolving the above component in 1 [symbol] of water, adding the activated carbon and zeolite having an average particle size of 2 to 7 mm at the normal temperature, dehydrating the resultant, and then drying the resultant at the temperature of about 90 to 120° C. for rapid drying without changing the shape of the activated carbon.

The deodorant 652 may include 5 to 15 parts by weight of zeolite based on 100 parts by weight of the activated carbon.

In the present invention, the reason for adding the above components to the activated carbon and zeolite at the normal temperature is that the pore structure of the activated carbon may be changed when the above components are dissolved and added at the high temperature, and particles that may reduce the surface area of the activated carbon may be formed when the cooled components are returned to the normal temperature.

The zeolite is a kind of feldspar mineral, in which water molecules are fully filled in nano-sized pores formed inside the zeolite. When the zeolite is heated, since the zeolite is an inorganic substance, the ore is not boiled, but the water molecules contained in the zeolite evaporate while generating the water vapor. This situation looks like boiling, so it is named to have the meaning of Greek [“boiling (zeo) stone (lithos)”].

The zeolite is now defined as [crystalline aluminum silicate having a cage or channel structure] through various verifications. The zeolites are widely used as catalysts, adsorbents, additives for detergents, additives for feed, and soil conditioners and it is known that there are a total of 200 species, both natural and synthetic.

In general, the phenomenon in which the concentration of a specific substance increases at the interface is called adsorption. When the concentration increases evenly throughout the total area as well as the interface, it is called absorption which is distinguished from adsorption. In a solid with very small pores densely developed, it can be said that the adsorbate is adsorbed to the surface when looking at the pore surface as an interface, but when viewed as a whole, the adsorbate is evenly spread across the interface. When it is difficult to distinguish between adsorption and absorption, it is called sorption. Most of the adsorption phenomena observed in the zeolites may be classified as sorption, but it is conventionally referred to as adsorption.

The advantage of zeolites is that they are stable at the high temperature. The activated carbon or charcoal has excellent porosity, but has the disadvantage that it is burnt when the temperature exceeds 400° C., whereas the zeolite has the advantage that it does not burn and the adsorbed material can be easily desorbed and new material can be adsorbed again through the heat treatment. The specific surface area, which is important for adsorption, is also quite high, such as natural zeolite: 300-600 m2/g, and synthetic zeolite: 800-900 m2/g, compared to charcoal: 250-300 m2/g and activated carbon: 800-1000 m2/g. In the present embodiment, the deodorant 652 contains the synthetic zeolite.

Hereinafter, an example of the operation principle of the food waste dryer 100 having the above-described configuration will be described.

First, when the front door 20 is closed and the switch unit 12 is operated in a state in which the food waste F is accommodated in the drying basket 23, external power is supplied through an electric wire extending from the rear of the body 10 to drive the air circulation unit 30 mounted in the drying space 11 inside the body 10.

The air generated by the driving of the impeller 32 contains the heat generated from the heater 33 and is transferred to the blower pipe 41, and then blown to the food waste F contained in the drying basket 23 through the blower pipe 41 to dry the food waste.

In this process, the dielectric sensor (not shown) measures the change in dielectric constant of the food waste dried by hot air to find out the amount of water vapor generated in the drying space 11, and controls the operation of the heater 33 and the impeller 32.

The air containing the water vapor vaporized and evaporated in the drying process through the heater 33 is supplied to the exhaust pipe 42 by air circulation of the circulation pipe 40, and the air containing the water vapor and supplied to the exhaust pipe 42 is transferred to the deodorizing device 60 mounted on the rear of the body 10 and adsorbed onto the deodorant 652 accommodated in the deodorizing device 60. The replacement cycle of the deodorant 652 is at least 3 months or more, usually about 6 months.

Accordingly, the clean air in which the odor is removed by the deodorant 652 is exhausted into the atmosphere through the mesh member 651 located at the upper end of the filter 65.

The food waste dryer 100 having the above configuration includes: a body 10 in which a drying space 11 is formed so that a portion thereof is open to an outside; a front door 20 for closing the open portion of the body 10; a drying basket 23 which is disposed in the drying space 11 of the body 10 and in which food waste is accommodated; an air circulation unit 30 including a heater adapted for heating air inside the drying space 11, in which the air circulation unit 30 suctions the air inside the drying space 11 and then blows the air heated by the heater 33 to the food waste so as to dry the food waste; a circulation pipe 40 configured to discharge air that includes water vapor vaporized during a food waste drying process to an outside through an exhaust pipe 42, and to recirculate air heated by the heater 33 to the drying space 11 through a blower pipe 41; and a deodorizing device 60 mounted on an outside of the body 10 so as to deodorize air by adsorbing odor particles from the air discharged to the outside through the circulation pipe 40, wherein the deodorizing device 60 includes a deodorant 652 including activated carbon and zeolite, so that the amount of water vapor vaporized and evaporated during the drying process for the food waste can be constantly maintained, and the adsorption and deodorization performance for the odor particles can be improved by the deodorant 652.

Further, in the food waste dryer 100, the ratio of the flow rate of air recirculated to the drying space through the blower pipe 41 to the flow rate of air discharged to the outside through the exhaust pipe 42 is set to 1:3 to 6, so the drying performance for the food waste F can be maximized by optimizing the amount of heat transferred to the food waste F and the processing efficiency of the deodorizing device 60.

In addition, according to the food waste dryer 100, since the deodorant 652 contains 5 to 15 parts by weight of zeolite based on 100 parts by weight of activated carbon, the deodorizing effect can be increased compared to the case where only the activated carbon is used.

Further, according to the food waste dryer 100, the deodorant 652 includes the activated carbon containing copper (Cu), manganese (Mn), sodium hydroxide (NaOH), and potassium hydroxide (KOH), there is an advantage that the removal efficiency for the acid odor can be increased compared to the case where only the activated carbon is used.

In addition, according to the food waste dryer 100, the deodorant 652 can be obtained by dissolving 5,000 to 80,000 mg of manganese (Mn), 5,000 to 200,000 mg of copper (Cu), 100 to 10,000 mg of sodium hydroxide (NaOH), and 100 to 10,000 mg of potassium hydroxide (KOH) in 1 [symbol] of water, adding the activated carbon having an average particle size of 0.5 to 1.5 mm and the zeolite having an average particle size of 2 to 7 mm at the normal temperature, dehydrating the resultant, and drying the resultant at the temperature of 90 to 120° C., the pore structure of the activated carbon does not change during the manufacturing process, and the surface area of the activated carbon does not decrease.

In addition, since the food waste dryer 100 may include the UV lamp 50 installed inside the body 10 to sterilize the food waste, various bacteria that may exist in the food waste F may be eliminated before the deodorizing process.

In addition, the food waste dryer 100 may include a temperature sensor (not shown) capable of detecting the temperature in the drying space 11, and when the temperature in the drying space 11 detected by the temperature sensor exceeds a predetermined temperature, the operation of the heater 33 may be stopped so that there is an advantage of preventing overheating of the heater 33 and minimizing the use of electric energy.

Although the present invention has been described above, the technical scope of the present invention is not limited to the contents described in the above-described embodiments, and it can be understood that the equivalent configuration modified or changed by those skilled in the art does not depart from the scope of the technical spirit of the present invention.

1. A food waste dryer comprising:
a body in which a drying space is formed so that a portion thereof is open to an outside;
a front door for closing the open portion of the body;
a drying basket which is disposed in the drying space of the body and in which food waste is accommodated;
an air circulation unit including a heater adapted for heating air inside the drying space, in which the air circulation unit suctions the air inside the drying space and then blows the air heated by the heater to the food waste so as to dry the food waste;
a circulation pipe configured to discharge air that includes water vapor vaporized during a food waste drying process to an outside through an exhaust pipe, and to recirculate air heated by the heater to the drying space through a blower pipe; and
a deodorizing device mounted on an outside of the body so as to deodorize air by adsorbing odor particles from the air discharged to the outside through the circulation pipe,
wherein the deodorizing device includes a deodorant including activated carbon and zeolite.

2. The food waste dryer of claim 1, wherein a ratio of a flow rate of the air recirculated to the drying space through the blower pipe to a flow rate of the air discharged to the outside through the exhaust pipe is 1:3 to 6.

3. The food waste dryer of claim 1, wherein the deodorant includes 5 to 15 parts by weight of zeolite based on 100 parts by weight of activated carbon.

4. The food waste dryer of claim 1, wherein the deodorant includes activated carbon containing copper (Cu), manganese (Mn), sodium hydroxide (NaOH), and potassium hydroxide (KOH).

5. The food waste dryer of claim 4, wherein the deodorant is obtained by dissolving 5,000 to 80,000 mg of manganese (Mn), 5,000 to 200,000 mg of copper (Cu), 100 to 10,000 mg of sodium hydroxide (NaOH), and 100 to 10,000 mg of potassium hydroxide (KOH) in 1 [symbol] of water, adding activated carbon having an average particle size of 0.5 to 1.5 mm and zeolite having an average particle size of 2 to 7 mm at a normal temperature, and dehydrating and drying the resultant at a temperature of 90 to 120° C.

6. The food waste dryer of claim 1, further comprising a UV lamp installed inside the body to sterilize the food waste.

7. The food waste dryer of claim 1, further comprising a temperature sensor adapted for detecting a temperature of the drying space, wherein an operation of the heater is stopped when the temperature of the drying space detected by the temperature sensor exceeds a predetermined temperature.
